(19) United States
(12) Patent Application Publication
(10) Pub. No.: US 2015/0289867 A1
(43) Pub. Date: Oct. 15, 2015

(54) NEEDLE FOR A LAPROSCOPIC INSTRUMENT

(71) Applicant: Azad Najar, Vasteras (SE)

(72) Inventor: Azad Najar, Vasteras (SE)

(73) Assignee: LAPROTECH AB, Vasteras (SE)

(21) Appl. No.: 14/402,712

(22) PCT Filed: May 23, 2012
(86) PCT No.: PCT/EP2012/059570

§ 371 Date: Apr. 28, 2015

(51) Int. Cl. A61B 17/04; A61B 17/062; A61B 17/06

(52) U.S. Cl. CPC A61B 17/0469; A61B 17/06109; A61B 17/062; A61B 2017/047; A61B 2017/06019; A61B 2017/06042; A61B 17/06004

(57) ABSTRACT

The present invention relates to a needle to be used with a laparoscopic instrument, comprising a proximal end arranged with a pointed tip (50) which may be pushed through tissue, and a suture thread holding member (52).

### TECHNICAL AREA

The present invention relates to a needle for a laparoscopic instrument and in particular a needle with which a suture thread may be introduced through tissue.

### BACKGROUND OF INVENTION

During a laparoscopic operation instruments with long handles are used in order to perform all steps of the operation. As a summary, the most important sewing-steps in a laparoscopic operation are the following: a first instrument holds the needle. The needle and the hanging thread are on one side of the tissues. With the help of the first instrument the needle is guided through the tissues that are to be sewn together. A second instrument receives the needle and then the first instrument releases the needle. The second instrument pulls the needle through the tissues so that the needle goes through from one side to a second side. The second instrument releases the needle and instead grips the thread at a suitable distance from the tissue on the second side. With suitable movements the thread is twisted around the first instrument. Then the first instrument grips the thread on the first side of the tissue. The first instrument pulls the end of the thread back so that a knot is created. Both instruments are pulled so that the tissues are brought together. A similar process is repeated two or more times so that at least three knots on each other are obtained so that the risk of them loosing the grip is minimized.

As understood from the above description, to make a knot during a laparoscopic operation is one of the most time-consuming parts of the operation. Limited possibilities of movement, two-dimensional viewing via a TV screen and the long handle of the instrument are some important factors that contribute to a higher degree of complexity. It is often that a knot may take more than ten minutes and the surgeon has to try several times before succeeding. Longer operation time is a large load for both the patient and the surgeon.

The applicant of the present invention has therefore developed a number of laparoscopic instruments that greatly facilitates a laparoscopic operation and thus shortens the operation time. These instruments are disclosed in the patent application, publication no. WO2007/073343. With the aid of these instruments both the entering of the thread through the tissues as well as the following knotting are handled much more easily than before.

However, even if the step of bringing the thread through the tissue with the above mentioned instruments has been reduced compared to before, there is still room for improvements in this area.

### BRIEF DESCRIPTION OF INVENTION

A main object of the present invention is to provide a needle, preferably but not exclusively intended for a laparoscopic instrument, that will further facilitate and shorten the operation time and in particular the step of bringing the thread through the tissue to be sutured.

This object is obtained by a needle according to the features of the independent patent claim. Preferable embodiments of the present invention form the subject of the dependent patent claims.

The main objective of the invention is to provide a needle which may be easily pushed through tissue to be sutured. In this respect the needle is provided with a proximal end having a pointed tip. The tip may have a circular cross-section, but may also have other cross-sectional shapes.

In order to perform a suture the needle is brought through the tissue with the pointed end making a passage through the tissue. Preferably the cross-sectional shape is circular but may also have other shapes.

The needle is further provided with a suture thread holding member. The suture thread holding member is arranged and designed to accommodate and hold at least one suture thread. The suture thread holding member may be arranged and designed to accommodate more than one suture thread if the application so requires.

In a preferred embodiment, the suture thread holding member is arranged as a groove or a cut-out on the needle into which the at least one suture thread may fit. Preferably the groove or cut-out is placed at a proximal part of the needle, more preferably at a part that predominantly is exposed after the needle has penetrated the tissue and most preferably adjacent the pointed tip of the needle.

The suture thread holding member can now be used to hold a suture thread when the needle is drawn back through the passage in the tissue, whereby the thread is drawn through the tissue. In most instances two parts of the suture thread is drawn through the tissue because the thread is often held and gripped at an area not too close to a thread end. When the thread has been drawn through the tissue it may then be arranged as a single thread or used as a double thread. The thread is then joined to form a knot in a suitable manner.

In one preferred embodiment, the groove or cut-out is positioned extending generally perpendicular to the longitudinal direction of the needle. However, other angles in relation to the longitudinal direction may also be used. The groove may further have a direction as seen in its depth direction that coincides with a transversal direction of the needle. As an alternative, the direction of the groove may be inclined with respect to the transversal direction of the needle.

In order to further facilitate the pulling back of the needle when the suture thread has been gripped, the proximal end of the needle may be arranged with a narrowed section such that the passage that has been created by the needle when penetrated the tissue now can house both the needle and the suture thread.

Further, the needle is preferably arranged with an attachment member at its distal end, which attachment member may cooperate with a corresponding attachment member on a laparoscopic instrument. In this manner it is possible to attach and replace a needle on the instrument. The attachment member may in one solution be the distal end itself, perhaps added with a planar surface on which a locking member may act. The attachment member may also be a support to which the needle is attached, where the support may be arranged with guides and the like for locking and attaching it to a proximal end of a laparoscopic instrument. The locking may be fixed as well as pivotal, the latter if it is desired to be able to direct the needle when inserted into a patient. This direction of the needle may be performed by suitable manoeuvring members on the handle of the laparoscopic instrument.

These and other aspects of and advantages with the present invention will become apparent from the following detailed description of the invention and from the accompanying drawings.

### BRIEF DESCRIPTION OF DRAWINGS

In the following detailed description of the invention, reference will be made to the accompanying drawings, of which

FIG. 1 is a perspective view of a laparoscopic instrument according to the invention,

FIG. 2 is a cross-sectional view taken in a longitudinal direction of the device of FIG. 1,

FIG. 3 is a detailed view of a proximal end part of the instrument of FIG. 1,

FIG. 4 is a detailed view of a needle and needle support comprised in the instrument of FIG. 1, and

FIG. 5 is an alternative embodiment of a laparoscopic instrument.

### DETAILED DESCRIPTION OF THE INVENTION

In the present application, when the term “distal part/end” is used, this refers to the part/end of needle or device to which the needle is associated, or the parts/ends of the members thereof, which is/are located the furthest away from the contact or operation site. Correspondingly, when the term “proximal part/end” is used, this refers to the part/end of the needle or device to which the needle is associated, or the parts/ends of the members thereof, which, is/are located closest to the contact or operation site.

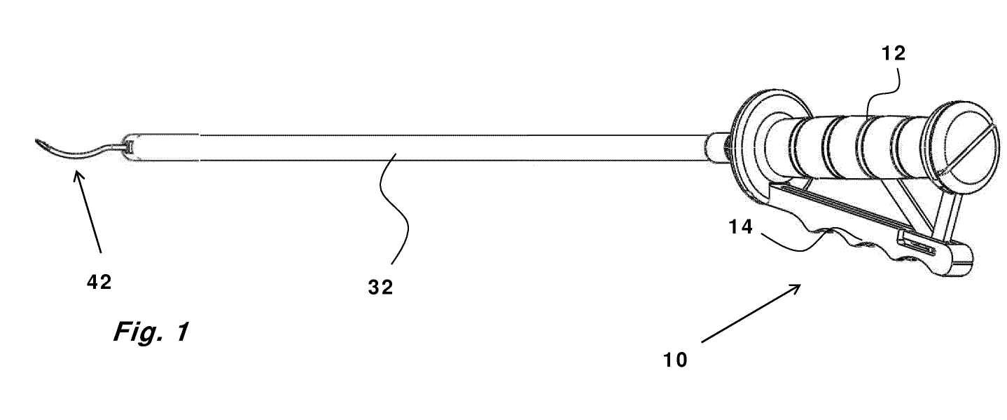

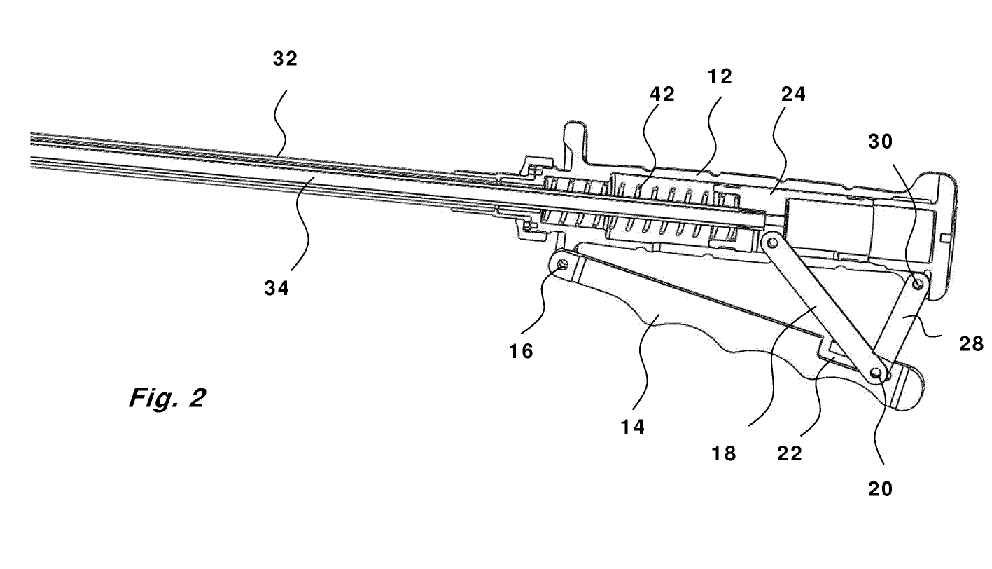

A laparoscopic instrument shown in FIGS. 1 and 2 comprises a manoeuvring part 10 that in the shown embodiment comprises a fixed handle part arranged as a hollow base support 12 and a movable handle part 14. The movable handle part 14 is pivotally attached to the base support 12 via a pivot axis 16, FIG. 2. To the distal end of the movable handle part 14, a first end of a first arm 18 is pivotally attached via a pivot axis 20. The pivot axis 20 is arranged slidable in a groove 22 provided in the movable handle part 14. A second end of said first arm 18 is pivotally attached to a generally tubularly shaped tool post 24, which tool post 24 is arranged sldable in the base support 12, via a pivot point 26. Further a second arm 28 is pivotally attached to the pivot axis 20 with a first end and attached to a distal end of the base support 12 via a further pivot point 30.

The base support 12 is further arranged with an elongated tubular insertion piece 32, which is intended to be introduced or inserted into the interior of a patient. Inside the insertion piece 32 an elongated manoeuvre rod 34, FIG. 2, is attached with a distal end to the tool post 24 so that the manoeuvre rod 34 also is slidable in the longitudinal direction.

The proximal end of the manoeuvre rod 34, FIG. 2, is pivotally attached to a needle attachment member 36 via a pivot axis 38, which needle attachment member 36 in turn is pivotally arranged to the proximal end of the tubular insertion piece 32. The needle attachment member 36 is thus capable of turning around its axis 38 when the proximal end of the manoeuvre rod 34 is moved in the proximal direction because the needle attachment member comes in contact with a rod 40, which rod 40 is arranged generally perpendicular to the longitudinal direction of the insertion piece 32. The needle attachment member 36 is arranged with a needle fixture. The needle fixture is arranged with an attachment orifice, into which a distal attachment end of a suture needle 42 may be inserted and locked by suitable means, such as a locking screw (not shown). Further, the proximal end of the insertion piece 32 has an opening 41 and is further provided with a first and a second orifice 43, 45, oppositely positioned.

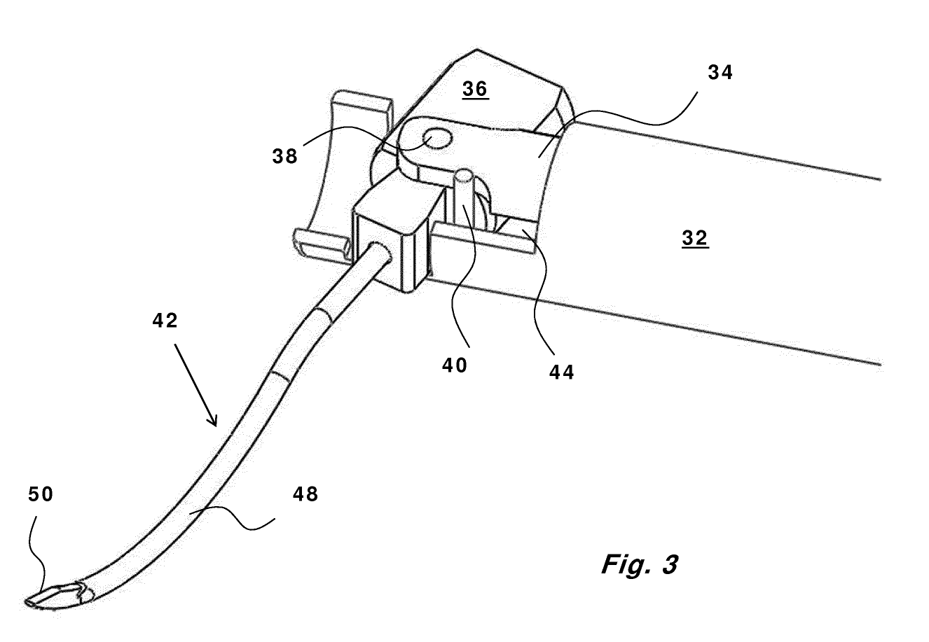

A longitudinally extending slit 44, FIG. 3, is arranged between the proximally directed opening 41 and the first orifice 43, through which slit 44 and first orifice 43 the needle 42 may protrude. The oppositely positioned second orifice 45 may accommodate the needle attachment member as will be described.

The needle 42 may be manoeuvred such with the help of the movable handle part 14 and the manoeuvre rod 34 that when the needle attachment member 36 is in a first position, the needle 42 is pointing generally in the longitudinal direction of the device through the proximally directed opening 41, thereby facilitating the insertion of the device into an orifice of a patient. In the first position, the movable handle 14 is not operated and the tool post 24 is in its most distal position, urged by a compression spring 46, FIG. 2, arranged between a proximal end surface of the tool post 24 and a distally directed wall of the base support 12. When the movable handle 14 is pressed by a user towards the fixed handle, the first arm 18 pivots and pushes the tool post 24 in the proximal direction against the force of the spring 42, whereby the manoeuvre rod 34 turns the needle attachment member 36 to a second position, FIG. 3, where the needle 42 is pointing generally perpendicular to the longitudinal direction after passing through the slit 44. In the second position the needle attachment member is moved into the second orifice 45 and is moved in contact with a distally directed side surface 47 of the second orifice, thereby locking the needle attachment member 36, and thus the needle 42, in that position. In this latter position, the needle may be used for creating stitches inside a patient.

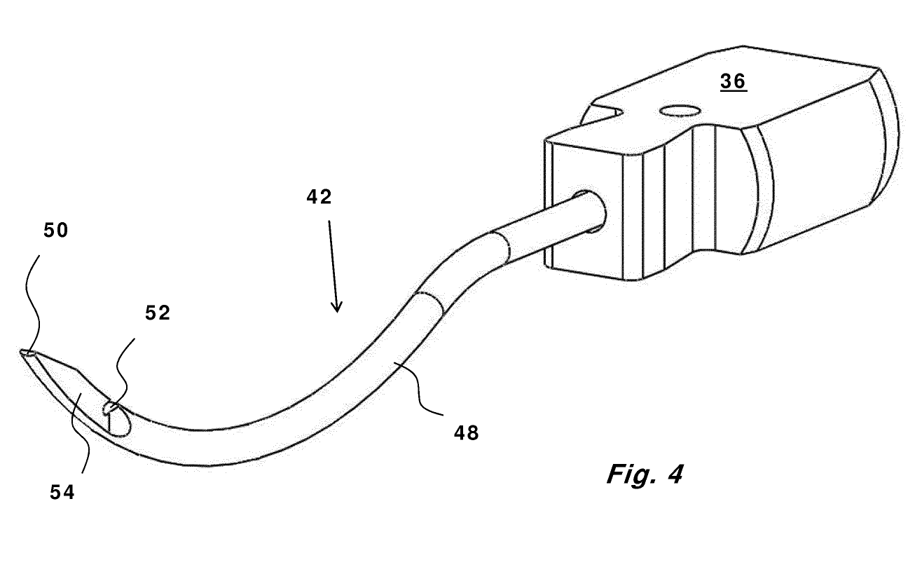

According to the invention a suture needle 42, FIG. 4, of a specific design is provided, having a distal attachment end insertable into the needle attachment member 36. The suture needle 42 is preferably arranged with a section 48, as seen in the longitudinal direction, having a somewhat curved shape for facilitating insertion into the tissue to be sutured. Further a proximal end of the suture needle 42 is provided with a sharpened pointed tip 50, also for facilitating entry of the needle through tissue. Further, the proximal end of the needle 42 is arranged with a suture thread holding member 52. This suture thread holding member 52 is in the embodiment shown a groove or a cut-out arranged and designed such that at least one suture thread may fit into the groove. Especially the groove or cut-out 52 should be designed such that it may hold a suture thread. Preferably the groove or cut-out 52 has an extension generally perpendicular to the longitudinal direction of the needle 42 in order to facilitate the holding of the suture thread. It is however to be understood that other angles may be chosen. Also the position of the groove on the needle as seen in a cross-sectional view in the longitudinal extension of the needle may be altered. If one considers that an upwards direction from the needle is 12 o'clock and downwards direction is 6 o'clock, then a suitable position for a right-handed user is between 7-8 o'clock and 4-5 o'clock for a left-handed person.

The main idea is that the needle is pushed, with the aid of the instrument, through the tissue to be sutured, where the pointed end penetrates the tissue. When the proximal part of the needle with the needle point and the suture thread holding member is through the tissue, a suture thread is placed in the groove 52 by another suitable instrument. The needle 42 is then drawn back through the tissue, whereby two parts of the thread are also drawn through the tissue. Preferably the thread placed in the groove has at least on free end, which free end is pulled through the tissue when the needle has been drawn back so that one part of the thread runs through the tissue. The both parts of the thread on each side of the tissue are now collected and joined to each other by a suitable method.

One such method of joining the suture thread parts that preferably may be used is disclosed in the patent application No. 1050737-4. There an instrument is used which may arrange a ring around the thread parts of a suture and to squeeze the ring such that the thread parts are locked to each other to form a stitch. It is however to be understood that other methods may be employed that are facilitating the making of a suture stitch in conjunction with the present invention.

It is also to be understood that the needle may have different cross-sections on parts of its extension in the longitudinal direction. For example the proximal end may in the vicinity of the needle tip 50 and the groove 52 be arranged with areas 54 with reduced cross-sectional area in order to facilitate the pulling back of the needle together with the suture thread through the tissue.

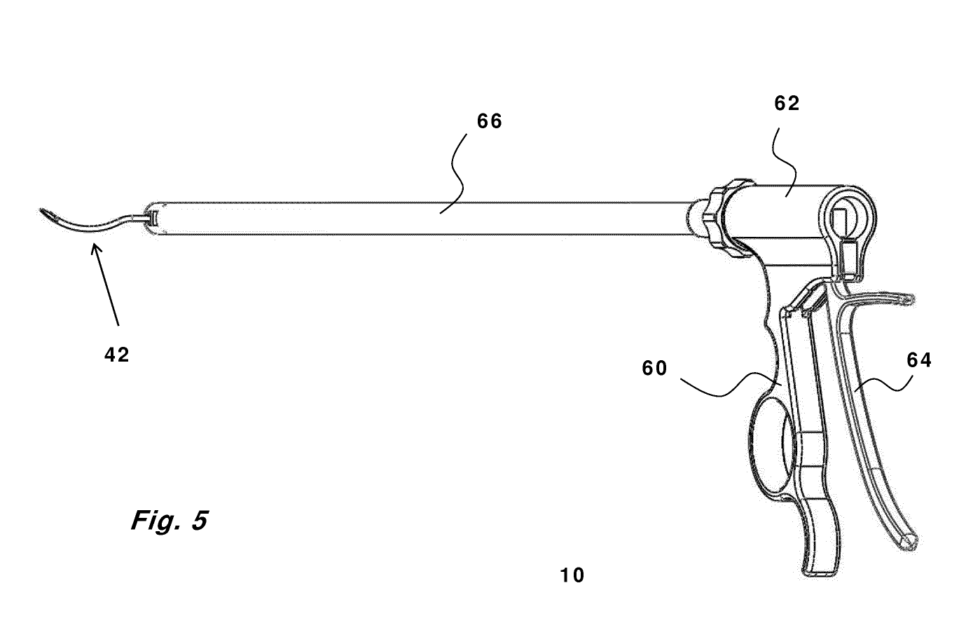

Further, the laparoscopic instrument may have a number of different designs that on the one hand may operate and manoeuvre the needle during suturing and on the other hand can enable and facilitate the attachment of a needle as well as replacement of a new needle. Another example of this is shown in FIG. 5 comprising a fixed handle 60 attached to a hollow base support 62 and a movable handle part 64. The movable handle part 64 is pivotally attached to the base support 62 such that an inner part of the movable handle part 64 is arranged inside the hollow base support 62. The base support 62 is further arranged with an elongated tubular insertion piece 66, which is intended to be introduced or inserted into the interior of a patient. Inside the insertion piece 66 an elongated manoeuvre rod and a needle attachment member (not shown) of the same design and function as described above are arranged. The function of the laparoscopic instrument according to FIG. 5 is the same as for the previous embodiment.

It is to be understood that the embodiment described above and shown in the drawings is to be regarded only as a non-limiting example of the invention and that it may be modified in many ways within the scope of the patent claims.

1. Needle to be used with a laparoscopic instrument, comprising:
a proximal end arranged with a pointed tip which can be pushed through tissue, and
a suture thread holding member.

2-14. (canceled)

15. The needle according to claim 1, wherein said suture thread holding member is arranged in the vicinity of the proximal end of the needle.

16. The needle according to claim 1, wherein said suture thread holding member comprises a groove or cut-out arranged and designed to house a suture thread.

17. The needle according to claim 16, wherein said groove or cut-out has an extension generally perpendicular to the longitudinal direction of the needle.

18. The needle according to claim 16, wherein said groove or cut-out has an extension angled in relation to the longitudinal direction of the needle.

19. The needle according to claim 15, wherein said pointed end is arranged with a reduced cross-sectional area as seen in the longitudinal direction of the needle, at least up to said suture thread holding member.

20. The needle according to claim 1, wherein the needle is further arranged with a section curved in the longitudinal direction.

21. The needle according to claim 1, wherein the needle is arranged with a distal end provided with a needle attachment member for attachment to a laparoscopic instrument.

22. A laparoscopic instrument according to claim 21, comprising a proximal end to be inserted into a patient, which proximal end is arranged with a complementary attachment member to the attachment member of the needle.

23. The laparoscopic instrument according to claim 23, comprising a grip with a movable handle part, which handle part is operably connected to said needle attachment member arranged at a proximal end of the instrument, capable, upon operation, of altering position of the needle in relation to the proximal end of the instrument.

24. The laparoscopic instrument according to claim 23, wherein said needle attachment member is arranged pivotally around a pivot axis at the proximal end of the instrument and that said handle part is capable of turning said needle attachment member around said pivot axis.

25. The laparoscopic instrument according to claim 24, wherein said needle attachment member is turnable between a first position wherein said needle is pointing generally in the longitudinal direction of the instrument, and a second position, wherein said needle is pointing generally perpendicular to the longitudinal direction of the instrument.

26. The laparoscopic instrument according to claim 25, wherein said needle attachment member is turnable around a shaft, which shaft is positioned generally perpendicular to the longitudinal direction of the instrument.

27. The laparoscopic instrument according to claim 26, wherein said attachment member is connected to a manoeuvre rod, which manoeuvre rod is attached to a manually operated handle at the distal end of the laparoscopic instrument.
